(19) United States
(12) Patent Application Publication
(10) Pub. No.: US 2011/0028561 A1
(43) Pub. Date: Feb. 3, 2011

(54) PROTECTION OF NEURAL RETINA BY REDUCTION OF ROD METABOLISM

(75) Inventors: James D. Akula, Boston, MA (US); Anne B. Fulton, Weymouth, MA (US); Ronald M. Hansen, Chelsea, MA (US)

Correspondence Address: DAVID S. RESNICK, NIXON PEABODY LLP, 100 SUMMER STREET, BOSTON, MA, 02110-2131, US

(73) Assignee: CHILDREN'S MEDICAL CENTER CORPORATION, Boston, MA (US)

(21) Appl. No.: 12/867,931

(22) PCT Filed: Feb. 23, 2009
(86) PCT No.: PCT/US09/34880

§ 371 Date: Oct. 19, 2010

Related U.S. Application Data
(60) Provisional application No. 61/030,681, filed on Feb. 22, 2008.

(51) Int. Cl. A61K 31/16; C12N 5/079; A61P 27/02

(52) U.S. Cl. 514/613; 435/375

(57) ABSTRACT

Described herein are methods for treatment or prevention of retinal dysfuntion by reduction of rod cell energy demand. Particular embodiments include methods for improving rod-mediated retinal function in a developing retina by administering an agent that reduces energy demand in the rod cell. Such agents that target rod cell energy demand can be administered to a subject at risk for retinal dysfunction to modify rod-cell function, such that the retina reaches retinal maturity. Also described herein are methods for suppressing the visual cycle in a developing rod cell by contacting the cell with an agent that reduces the energy demand of the rod cell.

### CROSS REFERENCE TO RELATED APPLICATION

This application claims priority to U.S. provisional application 61/030,681 filed Feb. 22, 2008, which is incorporated herein by reference in its entirety.

### GOVERNMENT SUPPORT

This invention was made with Government Support under Contract No. EY-10597 awarded by the National Eye Institute of the National Institutes of Health. The Government has certain rights in the invention.

### FIELD OF THE INVENTION

The invention relates to the treatment and/or prevention of retinal diseases or disorders.

### BACKGROUND OF THE INVENTION

Retinopathy of prematurity (ROP) blinds 400-800 babies annually in the USA alone, and reduces vision in many thousands more. It is a growing problem in the developing world. Fortunately, steady improvements in neonatal intensive care have led to an increase in the survival rate of very low birth weight infants. Unfortunately, these are the very patients at greatest risk for ROP. The retina contains specialized cells called photoreceptors that transduce light into a neural signal, and also has an extensive vascular supply. The clinical hallmark of ROP is abnormal retinal vasculature, which appears at the preterm ages characterized by maturation of the rod photoreceptors, cells that are the most demanding of oxygen of any cells in the body. In the most severe ROP cases, vision loss results from retinal detachment instigated by leaky retinal blood vessels. However, in the remaining cases of milder ROP, the retinal vascular abnormalities usually resolve without treatment, but the patients nevertheless suffer a range of lifelong visual impairments even with optimal optical correction.

### SUMMARY OF THE INVENTION

Described herein are methods and compositions for the treatment or prevention of diseases or disorders of the retina. More specifically, methods and compositions for the treatment or prevention of retinal diseases or disorders related to or involving vascular abnormalities. While the methods described herein can be applied to any of several different retinal diseases involving vascular abnormalities, retinopathy of prematurity is of particular interest. The methods described herein relate to the administration of compounds that reduce or suppress energy-demanding processes in rod photoreceptors. Improved management or prevention of ROP can be achieved through timely suppression of energy demanding processes in the rod photoreceptors. Compounds useful for suppression can include, for example, the vitamin A derivative N-retinylacetamide and derivatives thereof that retain the capacity to reduce or suppress energy demanding processes in rod photoreceptors. In a preferred embodiment, the N-retinylacetamide is all trans.

In one aspect, then, described herein is method of treating or preventing a retinal disease or disorder involving vascular abnormalities, the method comprising administering a compound that suppresses energy demand in rod photoreceptors of the eye. In one embodiment, the compound is N-retinylacetamide or a derivative thereof that suppresses energy demand in rod photoreceptors of the eye.

The retinal disease or disorder can be retinopathy of prematurity or other retinal diseases or disorders with a vascular component, e.g., age-related macular degeneration and diabetic retinopathy.

In another aspect, described herein is a method of improving rod-mediated retinal function, the method comprising administering to a subject with an immature retina, an agent that reduces rod energy demand in the developing retina, whereby rod-mediated retinal function is improved upon retinal maturity relative to a subject not treated with the agent.

In one embodiment of this aspect and all other aspects described herein, the subject is a premature infant.

In another embodiment of this aspect and all other aspects described herein, the subject is treated with supplemental oxygen.

Another aspect described herein relates to a method of treating or preventing a retinal disease or disorder involving vascular abnormalities, the method comprising administering an agent that suppresses energy demand in rod photoreceptors of the eye.

In one embodiment of this aspect and all other aspects described herein, the treatment comprises administering N-retinylacetamide or a derivative thereof that suppresses energy demand in rod photoreceptors of the eye.

In another embodiment of this aspect and all other aspects described herein, the treatment is administered locally to the eye.

In another embodiment of this aspect and all other aspects described herein, the treatment is administered at a site distant from the eye or systemically.

In another embodiment of this aspect and all other aspects described herein, the retinal disease or disorder is retinopathy of prematurity.

In another embodiment of this aspect and all other aspects described herein, the retinal disease or disorder is selected from age-related macular degeneration and diabetic retinopathy.

Also described herein is a method for improving function and/or suppressing the visual cycle in a developing rod cell, the method comprising contacting the cell with an agent that suppresses energy demand in the rod cell.

In one embodiment of this aspect and all other aspects described herein, the treatment comprises contacting the rod cell with N-retinylacetamide or a derivative thereof.

### DEFINITIONS

As used herein, the term “improving rod-mediated retinal function” refers to an increase in rod-mediated retinal function of at least 10%, preferably the increase is at least 20%, at least 30%, at least 40%, at least 50%, at least 60%, at least 70%, at least 80%, at least 90%, at least 95%, at least 99%, at least 1-fold, at least 2-fold, at least 5-fold, at least 10-fold, at least 50-fold, at least 100-fold, at least 1000-fold or higher. The term “rod-mediated retinal function” refers to a function of rod cells in a functioning retina and can include such clinical end-points as degree of peripheral vision, low-level light vision, scotopic/“night vision”, and sensitivity to peripheral movement. Rod-mediated retinal function can be assessed in vivo by e.g., electroretinography measurement of rod activation of photo-transduction or deactivation of photo-transduction; recovery of the dark current following photobleaching; measurement of the ERG a-wave or b-wave; speed of recovery to photo-transduction; or rod-mediated response amplitudes. Methods for measuring rod-mediated retinal function are known in the art and/or explained herein in more detail in the Detailed Description and Examples sections.

As used herein, the term “immature retina” refers to a retina of a preterm infant or a retina of similar morphology/function to that of a pre-term infant retina. In one embodiment, an immature retina can be characterized by the presence of poorly developed or disorganized blood vessels with or without the presence of scar tissue. In general, a human preterm infant is one born at 37 weeks gestation, or earlier. Conversely, the term “retinal maturity” refers to a retina of a full-term infant or a retina of similar morphology/function to that of a full-term infant.

As used herein, the phrases “reduces rod energy demand” or “suppresses rod energy demand” refer to a reduction in oxygen demand of a rod cell of at least 10%; preferably the reduction of oxygen demand of a rod cell is at least 20%, at least 30%, at least 40%, at least 50%, at least 60%, at least 70%, at least 80%, at least 90% or more. In general, it is preferred that the oxygen demand of a rod cell is maintained below the level necessary to induce pathological angiogenesis (i.e., blood vessel growth) or vascular abnormalities.

As used herein, the term “vascular abnormalities” is used to refer to an abnormal or pathological level of vascular blood vessel growth (e.g., angiogenesis) or morphology (e.g., tortuosity) that does not permit proper development of the retina to “retinal maturity” as that term is used herein. One of skill in the art can titrate the amount of agent administered or the timing of administration to maintain the growth and morphology of blood vessels below that of pathological blood vessel growth as assessed by e.g., Laser Doppler Blood Flow analysis. In an alternative embodiment, the level of tortuosity of retinal blood vessels is used to assess the degree of pathological blood vessel morphology and/or growth. Methods for measuring tortuosity are further described herein in the Detailed Description and/or Examples section.

As used herein, the term “supplemental oxygen” refers to a concentration of oxygen above that of ambient air (i.e., ˜20-21%) that is necessary to maintain blood oxygen levels in a subject at a desired level. In general, supplemental oxygen is supplied in a clinical setting to maintain a blood oxygen level of 100% as assessed using e.g., transcutaneous oxygen monitoring. Monitoring blood oxygen levels and altering the level of “supplemental oxygen” to maintain e.g., a 100% blood oxygen level is a standard procedure in a clinical setting (e.g., a neonatal intensive care unit) and is well known to those of skill in the art of medicine.

As used herein the term “agent ” refers to any organic or inorganic molecule, including but not limited to, modified and unmodified nucleic acids such as antisense nucleic acids, RNAi agents such as siRNA or shRNA, peptides, peptidomimetics, aptamers, drugs, prodrugs, metabolite analogs, small molecules, and antibodies.

As used herein, the term “therapeutically effective amount” refers to the amount of an agent that is effective, at dosages and for periods of time necessary to achieve the desired therapeutic result, e.g., a suppression of rod cell energy demand. A therapeutically effective amount of the inhibitors described herein, or functional derivatives thereof, may vary according to factors such as disease state, age, sex, and weight of the subject, and the ability of the therapeutic compound to elicit a desired response in the subject. The effective amount of a given therapeutic agent will also vary with factors such as the nature of the agent, the route of administration, the size and species of the mammal to receive the therapeutic agent, and the purpose of the administration. A therapeutically effective amount is also one in which any toxic or detrimental effects of the therapeutic agent are outweighed by the therapeutically beneficial effects. The effective amount in each individual case may be determined empirically by a skilled artisan according to established methods in the art. In general, an inhibitor is determined to be “therapeutically effective” in the methods described herein if (a) measurable symptom(s) of, for example, vascular abnormalities, are reduced for example by at least 10% compared to the measurement prior to treatment onset, (b) the progression of the disease is halted (e.g., patients do not worsen or the vasculature stops growing pathologically, or (c) symptoms are reduced or even ameliorated, for example, by measuring a reduction in vessel number or tortuosity. Efficacy of treatment can be judged by an ordinarily skilled practitioner or as described herein in the Detailed Description.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

### BRIEF DESCRIPTION OF THE FIGURES

FIG. 1. Diagram of the neural retina and its vascular supplies (not to scale). The layers of the neural retina (ganglion cell, inner plexiform, inner nuclear, outer plexiform, outer nuclear) are indicated. Blood flow through the choroidal vessels is swift. The retinal vasculature, visible by ophthalmosocopy, lies among the ganglion cells on the vitreal surface of the retina and extends capillary networks deep into the postreceptor layers. The caliber of the retinal arterioles adjusts to perturbations in blood oxygen levels (“autoregulation”).

FIG. 2. Logistic growth curve showing human rhodopsin content (Fulton, A. B., J. et al., Invest. Ophthalmol. Vis. Sci., (1999) 40: 1878-1883) as a function of age. The arrow indicates the age of ROP onset in preterm infants (Palmer, E. A., et al. Ophthalmology, (1991) 98:1628-1640).

FIG. 3. Rat model of retinopathy of prematurity. (a) Scanning laser ophthalmoscope (SLO) images obtained using blue (488 nm) laser stimulation (Seeliger, M. W., et al., Vision Res., (2005) 45: 3512-9) after injection of fluorescein in 22 day old control and ROP rats. (Pigmented rats were used to facilitate SLO imaging.) The integrated curvature of each retinal arteriole is expressed as a proportion of the mean (ICA) in the control. The higher ICA value for the ROP rat reflects the greater tortuosity of its arterioles. The choroidal appearance is similar in the control and ROP fundi. (b) Sample electroretinographic (ERG) responses to full-field stimuli in control and ROP rats. Both rats were tested with the same flash intensities, as indicated. The vertical grey lines indicate the time at which the flash was presented.

FIG. 4. Arteriolar integrated curvature (ICA) and rod photoreceptor sensitivity (SROD) in infants with a history of ROP (Fulton, A. B., et al., Arch. Ophthalmol., (2001) 119: 499-505; Gelman, R., et al., Invest. Ophthalmol. Vis. Sci., (2005) 46(12): 4734-4738; Moskowitz, A., et al., Optometry & Vision Science., (2005) 82: 307-317; Fulton, A. B. and R. M. Hansen, Mol. Vis., (2006) 12: 548-549) and in rat models (Akula, J. D., et al. Invest. Ophthalmol. Vis. Sci., (2007) 48: 4351-9), plotted as percent of normal for age (SEM). In both the human and animal subjects, mean ICA is nearly two times higher in ROP, and rod sensitivity (SROD) is reduced by ˜25%.

FIG. 5. Rat models of ROP. Two models, the “50/10 model” and the “75 model”, are induced by exposing infant rats to alternating periods of relatively high and low ambient oxygen; room air is 21%. For both models the exposures are delivered at ages during which the rod photoreceptors are immature, as indicated by the low rhodopsin content. ERGs and fundus images are obtained longitudinally in infant (20.1 day old), adolescent (30.1 day old), and adult (60.1 day old) rats (grey bars).

FIG. 6. Arteriolar curvature (ICA) and photoreceptor sensitivity (SROD) in 50/10 model, 75 model, and control rats expressed as a proportion of the mean in controls (dotted lines). For each subject, ICA measured at ˜60 days is plotted as a function of that subject's SROD measured earlier, at ˜20 days. The solid line is a linear regression through all the data. Early rod sensitivity predicted vascular outcome (r=−0.332; P=0.024). Adapted from FIG. 8, Akula, J. D., et al. Invest. Ophthalmol. Vis. Sci., (2007) 48: 4351-9).

FIG. 7. Mean (SEM) rate of change in post-receptor sensitivity and arteriolar curvature (ICA) for 50/10 model, 75 model, and control rats. Adapted from FIG. 7, Akula et al. Akula, J. D., et al. Invest. Ophthalmol. Vis. Sci., (2007) 48: 4351-9).

FIG. 8. Key features of the experimental paradigm. The ambient oxygen and light cycle were tightly controlled and synchronized. Dosing with the visual cycle modulation (VCM) (Reg-NH₂) was designed to target the rapid growth phase of the developmental increase in rhodopsin in the retina.

FIG. 9. Electroretinographic data. (A) Rod-saturating electroretinographic records elicited with bright ‘white’ flashes (lines), and fits of the Hood and Birch formulation of the Lamb and Pugh model of the activation of photo-transduction (dashes) to the leading edge of the a-wave (symbols). The flash eliciting the blue trace was subsequently used as a probe of the dark current. (B) Rod sensitivity. (C) Amplitude of the saturated rod response. (D) A bright probe flash (PF) is presented at fixed intervals (green ticks) after a ‘green’ conditioning flash (CF) to rapidly drive the rods to saturation, thus manifesting the constituent rod current. (E) The time-course of the rod photo-response to the CF is derived. The time to 50% recovery, τ, is estimated by fit of a line (red dashes) to the recovery phase of the photo-response (filled symbols). (F) Time constant of the deactivation of photo-transduction. (G) Scotopic ERG records elicited with ‘green’ flashes. (H) ERG b-wave amplitude plotted as a function of log flash intensity. The Naka-Rushton function (eq. 3) is fit through b-wave amplitudes only to intensities before major intrusion of the a-wave (filled symbols). The flash intensity producing half-maximal b-wave amplitude, log a, is indicated. (I) Post-receptor sensitivity. (J) Amplitude of the saturated post-receptor response. (K) The PF is presented in the dark, and at 2 minute intervals following a rhodopsin bleaching stimuli. The time to 50% recovery of the dark-adapted PF amplitude, t₅₀, is derived by fit of eq. 4. (L) Time constant of dark-adaptation. In panels B, C, F, I, J and L, error bars are SEMs and points are slightly offset on the age axis for clarity.

FIG. 10. Blood vessel data. (A) Sample composite fundus photographs from adult VCM and vehicle treated rats taken at P60-62. The white borders were added to demark the boundaries of individual images and were not present in the original analysis of the retinal arterioles. The analysis was constrained to the posterior pole (black circle). (B) The arterioles as segmented by RISA (black segments). RISA requires a bifurcation, so for un-bifurcated vessels, one was added arbitrarily (tan segments) though these were irrelevant to the analysis. (C) Tortuosity of the retinal arterioles. The long and short dashed green lines are the mean and 95% prediction intervals for normal rats, respectively, derived from previously published (J. D. Akula, et al. Invest Ophthalmol Vis Sci (2007) 48:4351) and unpublished data. Note that the data are plotted at a gain of 100 (centiradians). (D) Change in TA between first (P20-22) and last (P60-62) observations. In panels C and D, error bars are SEMs and in C points are slightly offset on the age axis for clarity.

### DETAILED DESCRIPTION

Described herein are methods and compositions for the treatment or prevention of diseases or disorders of the retina, and particularly, methods and compositions for the treatment or prevention of retinal diseases or disorders related to or involving vascular abnormalities. While the methods described herein can be applied to any of several different retinal diseases involving vascular abnormalities, retinopathy of prematurity is of particular interest. The methods described herein relate to the administration of treatments or compounds that reduce or suppress energy-demanding processes in rod photoreceptors. The following describes various aspects related to the understanding and practice of the invention described herein.

As a system, the mammalian retina is vulnerable to diseases that affect the exquisitely balanced interplay of the neural retina and the vasculature that nourishes it. Visual loss occurs when this balance is disturbed. On one hand, diseases such as photoreceptor degenerations that primarily affect the neural retina also affect the retinal vasculature. On the other hand, diseases that are clinically characterized by abnormality in the choroidal or retinal vasculature, such as age related macular degeneration, diabetic retinopathy, and retinopathy of prematurity (ROP), also affect the retinal neurons. Thus, all such diseases fall within the broad group of hypoxic ischemic disorders of neural tissue. Photoreceptors, specialized cells that have the highest oxygen requirements of any cell in the body (Steinberg, R., Invest. Ophthalmol. Vis. Sci., (1987) 28: 1888-1903), are likely important in all hypoxic ischemic diseases of the retina.

In normal development, as the rod photoreceptors differentiate and begin to produce rhodopsin (the molecule responsible for the capture of light), their extraordinarily high oxygen demands render the retina hypoxic, driving the growth of the retinal blood vessels. However, in ROP, supplemental oxygen administered for the acute cardiopulmonary care of the prematurely born infant renders the retina hyperoxic, interrupting normal vascular growth and leaving the peripheral retina avascular. Upon cessation of the supplemental oxygen, the peripheral retina becomes hypoxic. Hypoxia instigates a molecular cascade that leads to the formation of the abnormal retinal blood vessels that are the ‘clinical hallmark’ of ROP. Worse, even though the premature infant is subjected to high ambient oxygen, immature lungs and other medical complications often lead to fluctuations in blood oxygen and consequently to episodes of both hypoxia and hyperoxia at the retina; photoreceptors are susceptible to both too much and too little oxygen. Because the developing neural retina and its vasculature are under cooperative molecular control, the vascular abnormalities of ROP must be related to the function of the neural retina. Recent studies have found that the degree of dysfunction of the rods in ROP predicts the degree of abnormality observed in the retinal vasculature, but not the other way around. While not wishing to be bound by theory, this is consistent with the rods being the ‘root cause’ of ROP. Therefore, neuroprotection of the retina provides a therapeutic approach. Specific interventions (some pharmaceutical, others environmental) that can lead to improved outcomes in ROP are disclosed herein. For example, pharmaceutical treatments that reduce the energy demand of the rod photoreceptors can reduce inappropriate vascular proliferation. Examples are discussed herein below. Alternatively, environmental treatments, including, but not limited to increasing the light to which a patient is exposed, are also contemplated. Due to the physiology of the rod photoreceptors, metabolic demand is highest in low light situations—thus, exposure to increased light can reduce metabolic demand and thereby mitigate the manifestation of ROP or related diseases or disorders of the retina. In addition, these approaches are contemplated as treatments for other diseases, such as diabetic retinopathy, age-related macular degeneration, and any other retinal degeneration that shares ‘neurovascular’ features with ROP.

### Vascular and Neural Diseases of the Retina

Despite advancements in the medical management of neovascular diseases of the retina, such as retinopathy of prematurity (ROP), diabetic retinopathy, and age-related macular degeneration, they collectively remain the leading cause of blindness worldwide. For ROP, the current state-of-the art medical management is photocoagulation of the peripheral vasculature, which carries its own negative consequences, and experimental approaches such as treatment with anti-angiogenic pharmaceuticals, that have unknown efficacy. Rod photoreceptors, because they are peculiar to the eye and have among the highest oxygen requirements of any cell in the body, likely play a role in hypoxic ischemic neovascular retinal diseases (G. B. Arden, et al., Br J Ophthalmol (2005) 89:764; A. B. Fulton et al., Doc Ophthalmol, (2008) In Press. DOI: 10.1007/s10633). Rat models of ROP provide a convenient in vivo system in which the relation of the photoreceptors to the retinal vasculature can be studied and manipulated.

Abnormal retinal function is a feature of neovascular retinal diseases. Thus, these diseases could also be classified, therefore, as neurovascular diseases (A. B. Fulton et al., Doc Ophthalmol, (2008) In Press). From the conventional perspective, vision loss in neovascular retinal disease results from blood vessel abnormalities. Indeed, the severity of lifelong retinal dysfunction that persists after the blood vessel abnormalities resolve is related to the severity of the antecedent vascular disease (A. B. Fulton, et al., Arch Ophthalmol (2001) 119:499). Data from rat models of ROP, however, show that dysfunction of the rod photoreceptors precedes the vascular abnormalities by which ROP is conventionally defined and predicts their severity (X. Reynaud, and C. K. Dorey, Invest Ophthalmol Vis Sci (1994) 35:3169; J. D. Akula, Invest Ophthalmol Vis Sci (2007) 48: 4351). Thus, despite the fact that abnormalities in vascular morphology are the main diagnostic criterion, ROP may be primarily a disorder of the neural retina with only secondary vascular abnormalities. Of note, the appearance of the vascular abnormalities that characterize acute ROP is coincident with developmental elongation of the rod photoreceptors' outer segments and accompanying increase in the retinal content of rhodopsin (G. A. Lutty et al., Mol Vis (2006) 12: 532; O. Dembinska, et al., Invest Ophthalmol Vis Sci (2002) 43:2481). Substances that alter the function of immature rods, but have no direct effect on retinal vasculature were used in the Examples described herein to test the hypothesis that the rods have a causal role in retinopathy of prematurity (ROP). The results confirmed the hypothesis and indicate that such substances can be used therapeutically to treat ROP.

### Rod Cell Physiology and Metabolism

The rods perform three linked, metabolically demanding processes: generation of the dark current, maintenance of the visual pigment (the visual cycle), and outer segment turnover, all of which ensue concomitant to developmental elongation of the rod outer segments (ROS) and increase of the rhodopsin content of the eye. The signal transduction mechanism of the rods is physiologically unique. In darkness, sodium and other cations intromitted through cyclic guanosine monophosphate (cGMP) gated channels in the ROS are expelled by pumps in the rod inner segment (RIS) so rapidly that a volume equal to the entire cytosol is circulated every half minute (W. A. Hagins, et al. Proc Natl Acad Sci USA (1989) 86:1224). The molecular cascade initiated by photon capture by rhodopsin following a flash of light and leading to a reduction of cGMP leads the dark current to decay following the form of a delayed Gaussian that can be described by an intrinsic amplification constant, A (T. D. Lamb, and E. N. Pugh, Jr., J Physiol (1992) 449: 719; E. N. Pugh, Jr., and T. D. Lamb, Biochim Biophys Acta (1993) 1141:111).

Following photon capture, rhodopsin's chromophore (retinol) undergoes an isomeric change which frees it from opsin and initiates phototransduction. Spent chromophore is passed from the ROS to the retinal pigment epithelium (RPE) where it undergoes a series of transformations before being returned to the ROS through the apical processes of the RPE as retinol again. There it becomes covalently linked to its active-site lysine in opsin, becoming rhodopsin again and completing the visual cycle (R. R. Rando, Chem Rev (2001) 101:1881). The rate-limiting step in the visual cycle mediated by the isomerohydrolase enzyme complex, RPE65 (G. Moiseyev, et al., Proc Natl Acad Sci USA (2005) 102:12413). Other byproducts of photo-transduction in the ROS are expelled through a process of circadian shedding of the ROS tips; each RPE cell phagocytizes thousands of disks shed from 30-50 embedded rods each day (R. W. Young, J Cell Biol (1967) 33:61). Controlled down-regulation of the visual cycle through targeted inhibition of RPE65 activity lowers the flux of retinoids through the ROS/RPE complex. This would render the rods less vulnerable to insult from hyperoxia and hypoxia (J. Wellard, et al., Vis Neurosci (2005) 22:501) by reducing their metabolic demands. It might also slow phagocytosis and thus lengthen the rod outer segments.

Translation from Animal Models to Patients

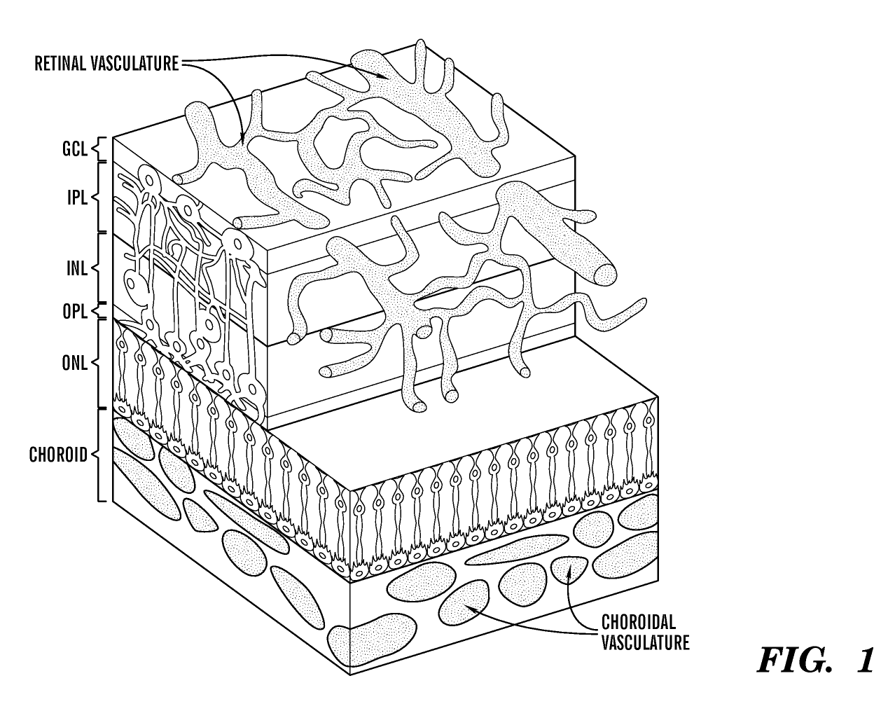

The photoreceptors are nestled closely to the choroidal vasculature (FIG. 1). Highly organized postreceptor retinal neurons form layers that are supplied by the retinal vessels. Although the choroid is the principal supply to the photoreceptors, degeneration of the photoreceptors is, nonetheless, associated with attenuation of the retinal arterioles (Hansen, R. M., et al., Vision Research (In press), (2007)). Because the photoreceptor layer is such an extraordinary oxygen sink, while not wishing to be bound by theory, it is presumed that, as photoreceptors degenerate, their metabolic demands wane and the retinal vasculature becomes attenuated consequent to the neural retina's chronic lower requirement for oxygen (Hansen, R. M., et al., Vision Research (In press), (2007)).

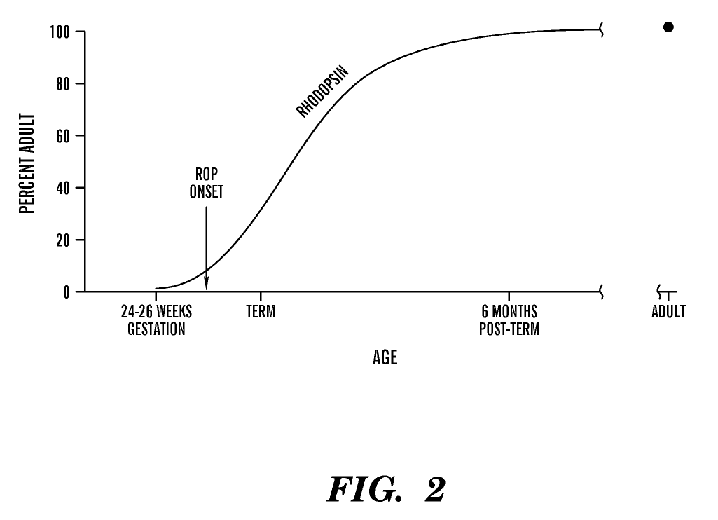

A tight link between the photoreceptors and the retinal vascular network is evident in the developing retina. Postreceptor cells differentiate before the photoreceptors, which are the last retinal cells to mature. As the formation of rod outer segments advances in a posterior to peripheral gradient, so too does vascular coverage. Thus, concurrent and cooperative development of the neural and vascular components characterize normal retinal maturation. In the preterm infant, the age of onset of ROP is around the age of rapid developmental increase in rod outer segment length and consequent increase in rhodopsin content (FIG. 2). This observation is circumstantial evidence of a role for the rod photoreceptors in the ROP disease process. In addition to immature photoreceptors and retinal vasculature, the preterm infant has immature lungs that create a precarious respiratory status with attendant risk of hypoxic injury to immature cells. This is countered by administration of supplemental oxygen. Both high and low oxygen levels are known to injure the immature photoreceptors (Fulton, A. B., et al. Invest. Ophthalmol. Vis. Sci., (1999) 40: 168-174; Wellard, J., et al., Vis. Neurosci., (2005) 22: 501-507).

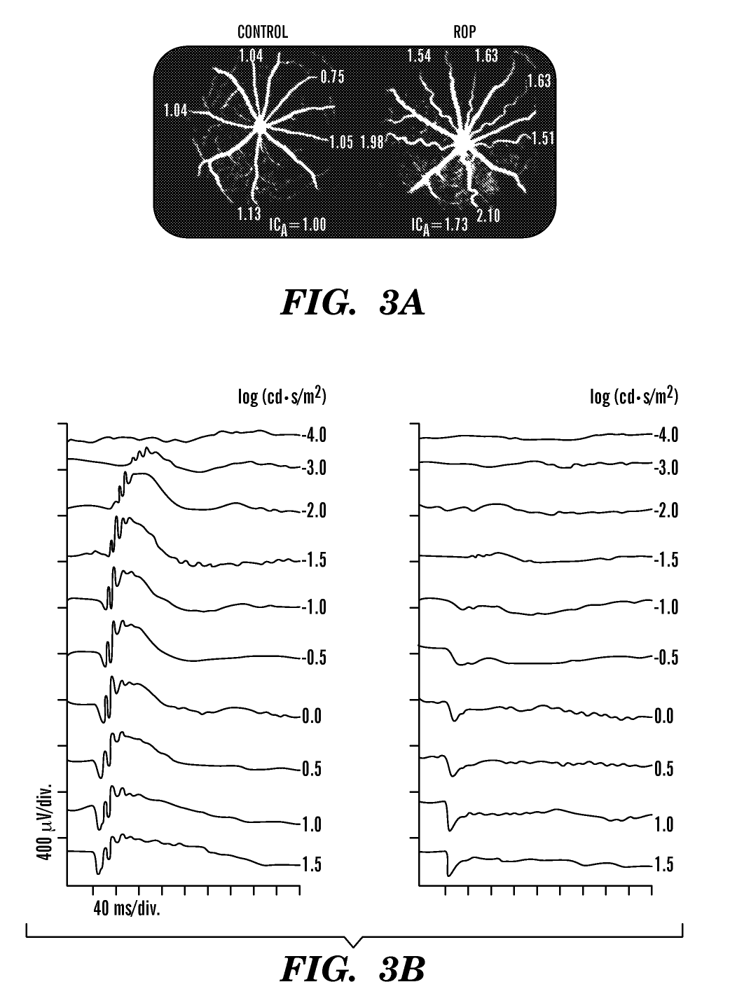

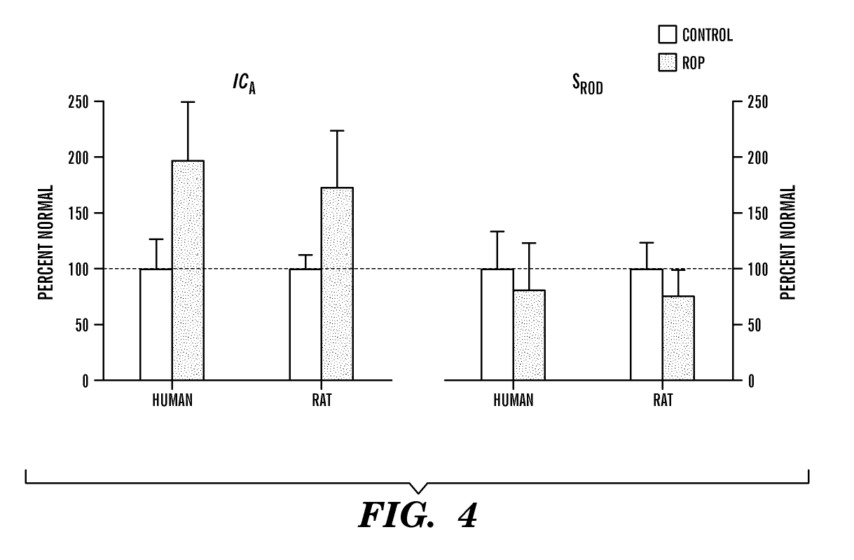

Indeed, rat models of ROP are induced by rearing pups in habitats with alternating periods of relatively high and low oxygen during the critical period of rod outer segment elongation (Akula, J. D., et al., Invest. Ophthalmol. Vis. Sci., (2007) 48: 4351-9; Akula, J. D., et al., Invest. Ophthalmol. Vis. Sci., (2007) 48: 5788-97; Dembinska, 0., et al., Invest. Ophthalmol. Vis. Sci., (2001) 42: 1111-1118; Liu, K., J. D. et al., Invest. Ophthalmol. Vis. Sci., (2006) 47: 5447-52; Liu, K., et al., Invest. Ophthalmol. Vis. Sci., (2006) 47: 2639-47; Penn, J. S., et al., Invest. Ophthalmol. Vis. Sci., 1995. 36: 2063-2070). Following induction, abnormalities of the retinal vasculature ensue, as do abnormalities of the structure and function of the neural retina (FIG. 3) (Fulton, A. B., et al. Invest. Ophthalmol. Vis. Sci., (1999) 40: 168-174; Akula, J. D., R. M. Hansen, et al., Invest. Ophthalmol. Vis. Sci., (2007) 48: 4351-9; Akula, J. D., et al., Invest. Ophthalmol. Vis. Sci., (2007) 48: 5788-97; Dembinska, O., et al, Invest. Ophthalmol. Vis. Sci., (2001) 42: 1111-1118; Liu, K., et al., Invest. Ophthalmol. Vis. Sci., (2006) 47: 5447-52; Liu, K., et al., Invest. Ophthalmol. Vis. Sci., (2006) 47: 2639-47; Reynaud, X., et al., Invest. Ophthalmol. Vis. Sci., (1995) 36:2071-2079). The abnormalities in the morphology of the retinal vasculature and in the function of the neural retina in ROP rats are similar to those found in pediatric ROP patients (FIG. 4) (Dembinska, O., et al., Invest. Ophthalmol. Vis. Sci., (2001) 42: 1111-1118; Liu, K., et al., Invest. Ophthalmol. Vis. Sci., (2006) 47: 5447-52; Liu, K., et al., Invest. Ophthalmol. Vis. Sci., (2006) 47: 2639-47; Reynaud, X., et al., Invest. Ophthalmol. Vis. Sci., (1995) 36:2071-2079; Barnaby, A. M., Invest. Ophthalmol. Vis. Sci., (2007). 48:4854-60; Fulton, A. B., et al., Arch. Ophthalmol., (2001)119: 499-505; Gelman, R., Invest. Ophthalmol. Vis. Sci., (2005) 46(12): 4734-4738; Moskowitz, A., et al., Optometry & Vision Science., (2005) 82: 307-317; Fulton, A. B., Invest. Ophthalmol. Vis. Sci., (2008) In press). Thus, translation from the rat models to the human condition are justified.

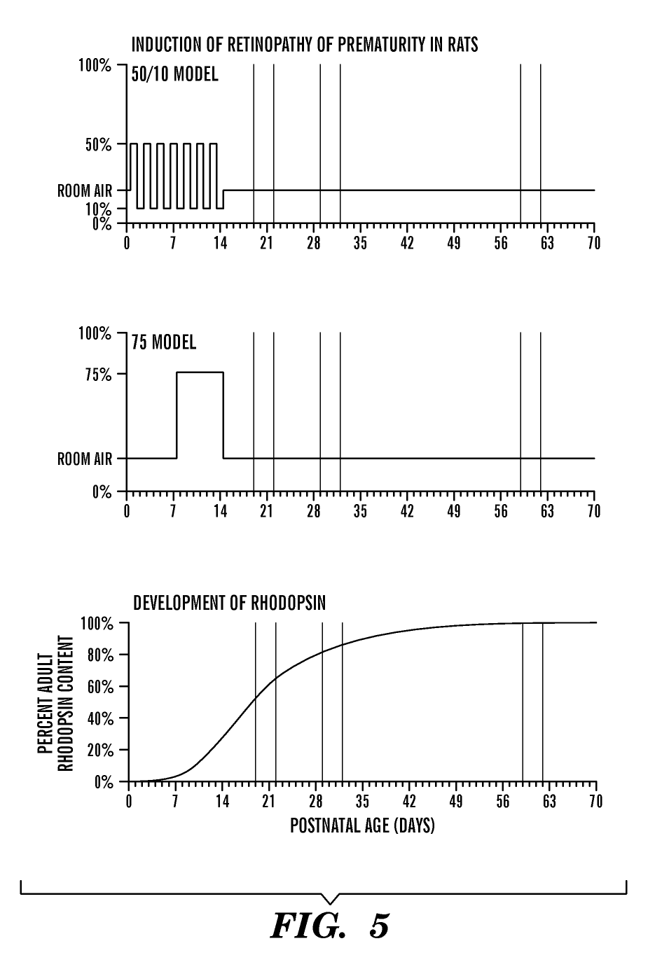

Albino rat models of ROP are used to study the neural and vascular characteristics of the retina during development (Akula, J. D., et al., Invest. Ophthalmol. Vis. Sci., (2007) 48: 4351-9; Akula, J. D., et al., Invest. Ophthalmol. Vis. Sci., (2007) 48: 5788-97; Liu, K., Invest. Ophthalmol. Vis. Sci., (2006) 47: 5447-52; Liu, K., et al., Invest. Ophthalmol. Vis. Sci., (2006) 47: 2639-47). Different schedules of oxygen exposure induce a range of effects on the retinal vasculature and the neural retina that model the gamut of retinopathy, mild to severe, observed in human ROP cases. As shown in FIG. 5, the oxygen exposures are timed to impact the retina during the ages when the rod outer segments are elongating and the rhodopsin content of the retina is increasing. Longitudinal measures of electroretinographic (ERG) responses and retinal vascular features are obtained in infant (˜20 day old), adolescent (˜30 day old), and adult (˜60 day old) rats. Results from longitudinal investigations in ROP rats indicate at least two retinal targets for drug therapies.

### Assessment of Neural Function

The ERG is used to characterize neural function. ERG responses to full-field stimuli over a range of intensities are recorded from the dark-adapted animal as previously described in detail (Akula, J. D., et al., Invest. Ophthalmol. Vis. Sci., (2007) 48: 4351-9). To summarize rod photoreceptor activity, a model of the activation of phototransduction is fit to the a-waves (Hood, D. C. and D. G. Birch, Invest. Ophthalmol. Vis. Sci., (1994) 35: 2948-2961; Lamb, T. D. and E. N. J. Pugh, J. Physiol. (Lond). (1992) 449: 719-758; Pugh, E. N., Jr. and T. D. Lamb, Biochim. Biophys. Acta, 1993. 1141: 111-149; Pugh, E. N., Jr and T. D. Lamb, in Handbook of biological physics. Volume 3 (2000), Elsevier Science. p. 183-255) and the resulting sensitivity (SROD) and saturated amplitude (RROD) parameters are calculated. Post-receptor activity is represented by the b-wave. The stimulus/response functions are summarized by the saturated amplitude (Vmax) and the stimulus producing a half-maximum response (log s); these parameters are derived from the Michaelis-Menten function fit to the b-wave amplitudes (Akula, J. D., et al., Invest. Ophthalmol. Vis. Sci., (2007) 48: 4351-9). Although significant deficits in the amplitude parameters (RROD and Vmax) are apparent, it is the sensitivity parameters (SROD and logs) that, as described in the following sections, direct the inventors to the described intervention sites.

### Assessment of Vascular Characteristics

Retinal vascular parameters are derived using image analysis software applied to digital fundus photographs (Akula, J. D., et al., Invest. Ophthalmol. Vis. Sci., (2007) 48: 4351-9; Martinez-Perez, M. E., (2001), Imperial College: London; Martinez-Perez, M. E., et al., Trans. Biomed. Eng., (2002) 49: 912-917). Integrated curvature (IC), which agrees well with subjective assessment of vascular tortuosity reported by experienced clinicians (Gelman, R., M. Invest. Ophthalmol. Vis. Sci., (2005) 46(12): 4734-4738), is used to specify the vascular status of each fundus. Both arterioles and venules are significantly affected by ROP. It was found, however, that the arterioles are markedly affected, while the venules are less so (Akula, J. D., et al., Ophthalmol. Vis. Sci., (2007) 48: 4351-9; Liu, K., et al., Invest. Ophthalmol. Vis. Sci., (2006) 47: 5447-52; Liu, K., et al., Invest. Ophthalmol. Vis. Sci., (2006) 47: 2639-47; Gelman, R., M. Invest. Ophthalmol. Vis. Sci., (2005) 46(12): 4734-4738); therefore, the arteriolar parameter ICA is used in the analyses described herein.

### Relation of Retinal Sensitivity and Vasculature

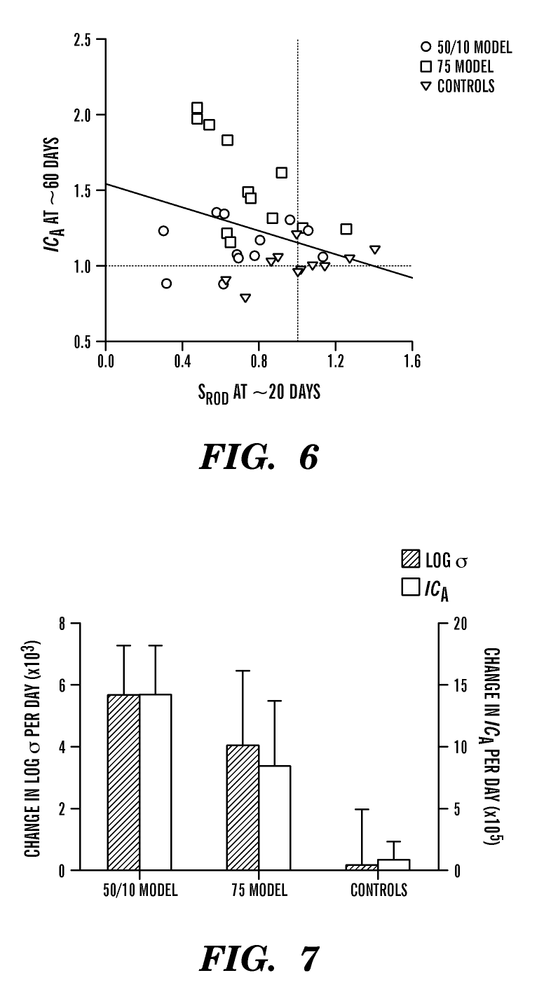

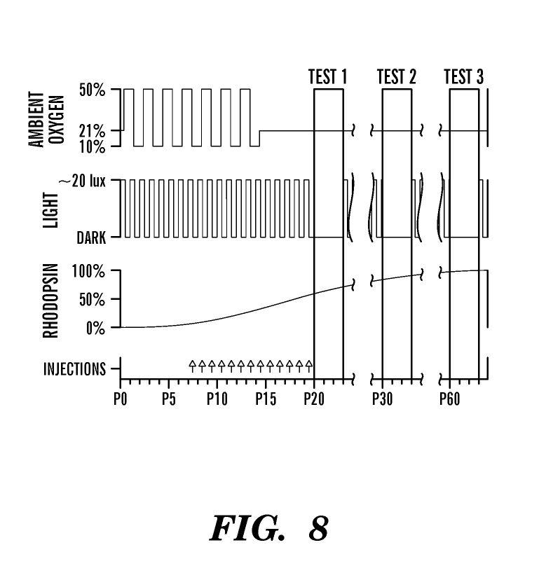

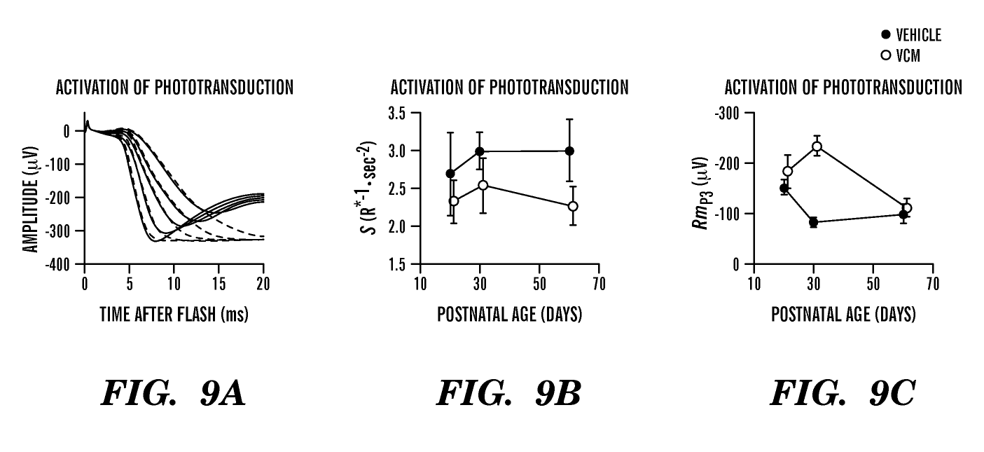

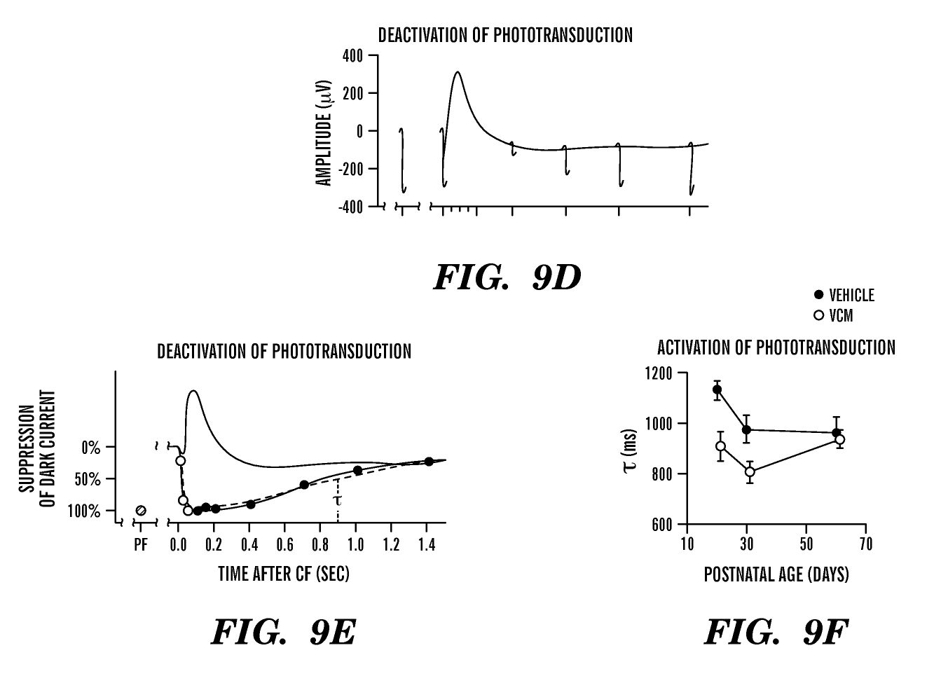

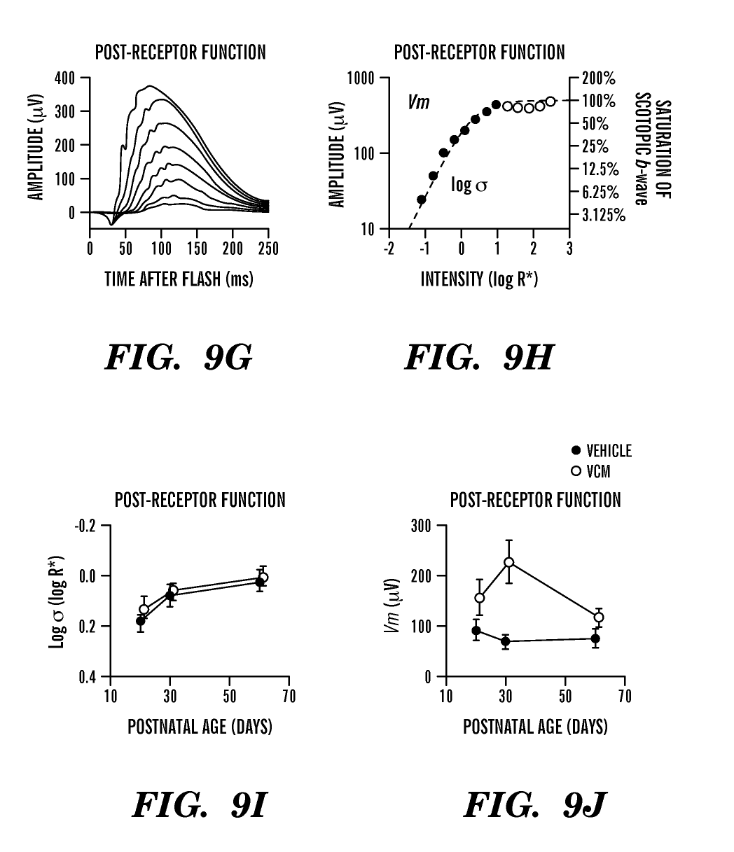

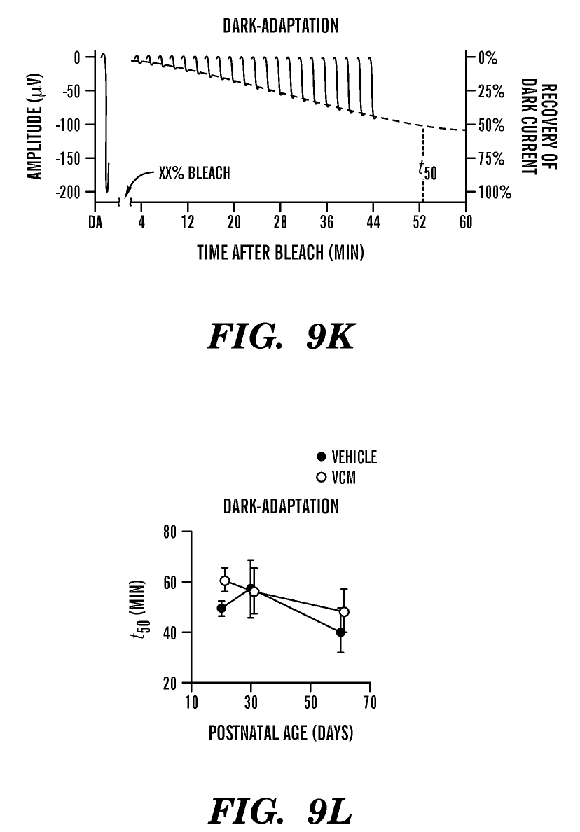

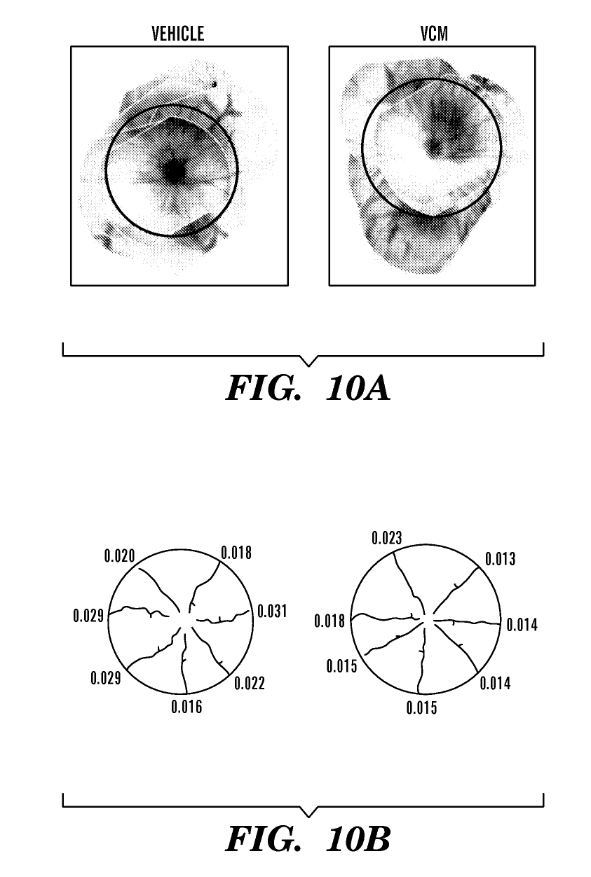

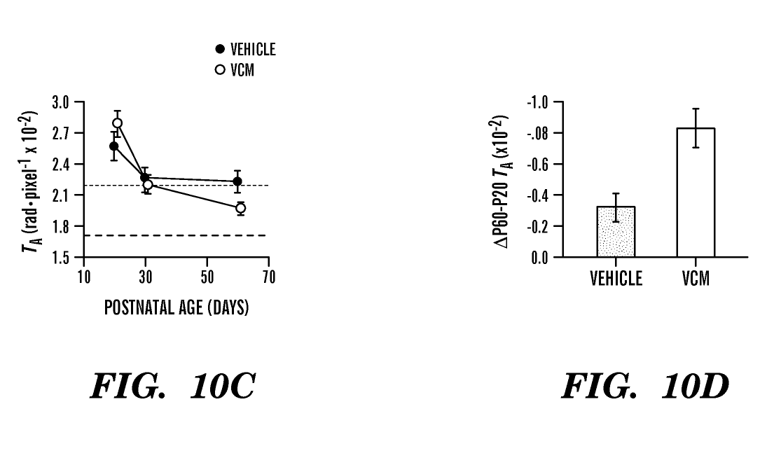

Rod photoreceptor sensitivity (SROD) at a young age (20 days) predicts retinal vascular outcome as specified by ICA. Better sensitivity at an early age is associated with better (less tortuous) vascular outcome (FIG. 6) (Akula, J. D., et al., Invest. Ophthalmol. Vis. Sci., (2007) 48: 4351-9). After cessation of the inducing oxygen exposure, recovery of postreceptor neural retinal sensitivity (b-wave log s) and decrease of vascular tortuosity occur hand-in-hand (FIG. 7). However, at a given age, sensitivity and vascular status are not correlated. Thus, simple compromise of vascular supply to the post-receptor neurons is, at most, an incomplete explanation for the low sensitivity. It is noted that the regulation of developing retinal neurons and blood vessels is complex, being under the cooperative control of several growth factors, such as vascular endothelial growth factor (VEGF), semaphorin, and their neuropilin receptors (Gariano, R. F., et al., Gene Expression Patterns, (2006) 6: 187-192). In rat models of ROP, expression of these growth factors is altered (Mocko, J. A. et al., ARVO Absract, (2008).

### Identification of Targets for Pharmaceutical Interventions

The longitudinal data from rat models of ROP identify at least two targets for pharmaceutical intervention: (1) the immature photoreceptors and (2) the molecular cross-talk between neurons and retinal vasculature. The rationale for each is outlined below. VEGF promotes development of retinal vasculature. Hypoxia promotes expression of VEGF. The rods are the most demanding of aerobic energy of any cells in the body [1]. Thus, rod-instigated hypoxia is believed to lead to up-regulation of growth factors that promote retinal vascular development. Low rod sensitivity at an early age predicts the poorer vascular outcome at older ages (FIG. 6). Because low rod sensitivity appears to be indicative of early injury to the rod (Fulton, A. B., et al., Invest. Ophthalmol. Vis. Sci., (1999) 40: 168-174; Reynaud, X., et al., Invest. Ophthalmol. Vis. Sci., (1995) 36: 2071-2079), therapy designed to relieve the immature rod of its burgeoning oxygen-based energy requirements is expected to be beneficial. This expectation is borne out in experiments described in the Examples provided herein. It is at the ages during which the developing rod outer segments elongate with consequent escalation of energy demands that ROP has its onset (FIG. 2). Arden and colleagues (Arden, G. B., Brit J Ophthalmol, (2005) 89: 764-769) proposed treatment with light to reduce the ROP rods' energy requirements by suppressing the circulating current. Light has a mild beneficial effect on a rat model of ROP (Fulton, A. B., et al., Invest. Ophthalmol. Vis. Sci., (1998) 39: S-820). Careful regulation of supplemental oxygen so that arterial oxygen levels are neither too high nor too low may also have some beneficial effect on ROP (Askie, L. M., N. Engl. J. Med., (2003) 349: 959-67; Vanderveen, D. K., et al., J. AAPOS, (2006) 10: 445-8; Chow, L. C., Pediatrics, (2003). 111: 339-45; Saugstad, O. D., J. Perinatol., (2006) 26 Suppl 1:S46-50; discussion S63-4). One can argue that optimal timing for light and oxygen interventions has yet to be specified, but, to date, adjustments in light and oxygen alone have not been a panacea for ROP. Thus, pharmaceutical approaches are also warranted. Improved management, or better yet, prevention of ROP can be achieved through timely pharmaceutical suppression of rods' energy demanding processes such as turnover of outer segment material (Tamai, M., Invest. Ophthalmol. Vis. Sci., (1982). 22: 439-48) and generation of the circulating current (Pugh, E. N., Jr. and T. D. Lamb, Biochim. Biophys. Acta, (1993) 1141: 111-149).

The second target for pharmaceutical intervention, the molecular cross talk between the retinal vasculature and postreceptor neurons, deserves attention first because abnormal retinal vasculature provides the usual clinical definition of ROP, and also because the postreceptor neurons have a great capacity to remodel (Jones, B. W., et al., J. Comp. Neurol., (2003) 464: 1-16) in the face of irreversible damage to the photoreceptors (Lu, M., Arch. Ophthalmol., (2007) 125(11):1581-1582). Damage to photoreceptors in ROP may be unavoidable as there is evidence of persistent damage to the rods even in mild cases (Fulton, A. B., et al., Arch. Ophthalmol., (2001) 119: 499-505; Reisner, D. S., et al., Invest. Ophthalmol. Vis. Sci., (1997) 38: 1175-1183). It is contemplated that treatment of the retinal vasculature can improve visual function (e.g., contrast sensitivity) by an associated beneficial effect on the postreceptor neurons. However, treatment of ROP by manipulating growth factors must be approached with considerable caution because the same growth factors regulate the developing photorececeptors and postreceptor retinal neurons (Yourey, P. A., et al., J. Neurosci., (2000) 20: 6781-8; Hashimoto, T., et al., Development, (2006) 133: 2201-10). Indeed, particular caution in use of anti-VEGF treatment in immature retina has been advised specifically because of the potential for adverse effects on the developing neurons (Nishiguchi, K. M., et al., Invest. Ophthalmol. Vis. Sci., (2007) 48: 4315-20).

A systems biology approach is applicable not only to animal models but also to the human retina. Such an approach is supported by the combination of full-field ERG analysis of the neural retina and image analysis of the retinal vasculature. Using the multifocal ERG and high resolution optical coherence tomography (OCT), the systems approach is being applied to the study of disease of the macula in ROP (Fulton, A. B., et al., Doc. Ophthalmol., (2005) 111: 7-13; Hammer, D. X., Invest. Ophthalmol. Vis. Sci., (2008), In press).

In summary, the perspective that is illustrated herein by study of ROP can also yield novel perspectives on other hypoxic ischemic retinal disorders, including diabetic retinopathy and age related macular degeneration, diseases which are characterized by both vascular and neural abnormalities of the retina. That is, treatment of these diseases can also be accomplished by therapies that reduce the metabolic demands of the neural retina.

### Compositions Effective for Therapy of Retinal Diseases or Disorders Involving Vascular Abnormalities:

As disclosed herein, compositions for the treatment of retinal diseases or disorders involving vascular abnormalities can include compounds that reduce the metabolic energy demands of rod photoreceptors of the eye. Among such compositions are, for example, the vitamin A derivative N-retinylacetamide, preferably all trans N-retinylacetamide, or derivatives thereof that retain the ability to reduce rod photoreceptor metabolic energy demand.

### Dosage, Administration and Efficacy:

Treatments described herein can be administered and monitored by an ordinarily skilled clinician. Administration routes, dosages and specific measures of efficacy can be selected by the administering clinician, and will depend upon factors such as the specific disease involved, severity of that disease, age, weight and gender of the patient, as well as other factors, such as other medical problems faced by the patient concurrently.

Efficacy for any given drug (e.g., N-retinylacetamide) or formulation of that drug can also be judged using an experimental animal model, e.g., the rat model of ROP described herein, When using an experimental animal model, efficacy of treatment is evidenced when a reduction in a marker or symptom of the retinal disease or disorder is observed.

In addition, the amount of a composition to be administered depends upon the frequency of administration, such as whether administration is once a day, twice a day, 3 times a day or 4 times a day, once a week; or several times a week, for example 2 or 3, or 4 times a week.

The amount and frequency of administration depends upon the compositions itself, its stability and specific activity, as well as the route of administration. Greater amounts of a composition will generally have to be administered for systemic, as opposed to topically administered drugs. However, as demonstrated in the Example herein, systemic administration by injection can be quite effective. The eye provides a tissue or structure well suited for topical administration of many drugs. Alternatively, intraocular injection can be effective. Doses will vary depending on route of administration, and will vary from, e.g., about 0.1 mg/kg body weight to about 10 mg/kg body weight for by systemic administration, to 0.01 mg to 10 mg by topical or intraocular injection routes.

While topical administration is preferred, oral administration or intravenous administration can also be used. Solid dosage forms for oral administration include, for example but not limited to capsules, tablets, pills, powders and granules. In such solid dosage forms, the compositions as disclosed herein may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The active components can also be in micro-encapsulated form, if appropriate, with one or more of the above-mentioned excipients. In the preparation of pharmaceutical formulations as disclosed herein in the form of dosage units for oral administration the compound selected can be mixed with solid, powdered ingredients, such as lactose, saccharose, sorbitol, mannitol, starch, arnylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture is then processed into granules or pressed into tablets.

In addition, compositions for topical (e.g., ocular, oral mucosa, respiratory mucosa) and/or oral administration can form solutions, suspensions, tablets, pills, capsules, sustained-release formulations, oral rinses, or powders, as known in the art are described herein. The compositions also can include stabilizers and preservatives. For examples of carriers, stabilizers and adjuvants, see, e.g., University of the Sciences in Philadelphia (2005) Remington: The Science and Practice of Pharmacy with Facts and Comparisons, 21st Ed.

The compositions described herein for reducing metabolic demand of neural retina can also be administered in conjunction with one or more additional drugs or therapeutics if so desired.

Liquid dosage forms for oral administration include pharmaceutically acceptable emulsions, solutions, suspensions, syrups and elixirs. In addition to the active components, the liquid dosage forms may contain inert diluents commonly used in the art such as, for example, water or other solvents that are compatible with the maintenance of drug in solution or soluble form. Liquid preparations for oral administration can also be prepared in the form of syrups or suspensions, e.g. solutions or suspensions containing from 0.2% to 20% by weight of the active ingredient and the remainder consisting of sugar or sugar alcohols and a mixture of ethanol, water, glycerol, propylene glycol and polyethylene glycol provided that such solvent is compatible with maintaining the micelle form. If desired, such liquid preparations can contain coloring agents, flavoring agents, saccharin and carboxymethyl cellulose or other thickening agents. Liquid preparations for oral administration can also be prepared in the form of a dry powder to be reconstituted with a suitable solvent prior to use.

Besides inert diluents, the oral compositions may also include adjuvants such as wetting agents, emulsifying and suspending agents, sweetening, flavoring and perfuming agents

Where an important target population is premature infants, oral dosage is not necessarily preferred. For these instances, topical administration and/or systemic administration by, e.g., intravenous routes is preferred.

Transdermal patches can also be used to provide controlled delivery of the formulations and compositions as disclosed herein to specific regions of the body. Such dosage forms can be made by dissolving or dispensing the component in the proper medium. Absorption enhancers can also be used to increase the flux of the compound across the skin. The rate can be controlled by either providing a rate-controlling membrane or by dispersing the compound in a polymer matrix or gel.

As noted, an important delivery route includes topical administration to the eye. Compositions described herein can be delivered, e.g., in a pharmaceutically acceptable ophthalmic vehicle, such that the component is maintained in contact with the ocular surface for a sufficient time period to allow the component to penetrate the corneal and internal regions of the eye, as for example the anterior chamber, posterior chamber, vitreous body, aqueous humor, vitreous humor, cornea, iris/cilary, lens, choroid/retina and sclera. The pharmaceutically acceptable ophthalmic vehicle may, for example, be an ointment or an encapsulating material.

In an alternative embodiment, the compositions and formulations as disclosed herein can be also administered via rectal or vaginal administration. In such embodiments, the compositions and formulations as disclosed herein can be in the form of suppositories which can be prepared by mixing the compounds with suitable non-irritating excipients or carriers such as cocoa butter, polyethylene glycol or a suppository wax which are solid at room temperature but liquid at body temperature and therefore melt in the rectum or vaginal cavity and release the active component.

Alternatively, compositions and formulations as disclosed herein can be in a form of enteric- coated preparation for oral administration. In some embodiments, a drug-containing core for coating with an enteric coating film can be prepared using an oleaginous base or by other known formulation methods without using an oleaginous base. In some embodiments, the compositions and formulations as disclosed herein in the form of the drug-containing core for coating with a coating agent may be, for example, tablets, pills and granules.

The excipient contained in the core is exemplified by saccharides, such as sucrose, lactose, mannitol and glucose, starch, crystalline cellulose and calcium phosphate. Useful binders include polyvinyl alcohol, hydroxypropyl cellulose, macrogol, Pluronic F-68, gum arabic, gelatin and starch. Useful disintegrants include carboxymethyl cellulose calcium (ECG505), crosslinked carboxymethylcellulose sodium (Ac-Di-Sol), polyvinylpyrrolidone and low-substituted hydroxypropyl cellulose (L-HPC). Useful lubricants and antiflocculants include talc and magnesium stearate.

The enteric coating agent can be an enteric polymer which is substantially insoluble in the acidic pH and is at least partially soluble at weaker acidic pH through the basic pH range. The range of acidic pH is about 0.5 to about 4.5, preferably about 1.0 to about 2.0. The range of weaker acidic pH through basic pH is about 5.0 to about 9.0, preferably about 6.0 to about 7.5. Specifically, cellulose acetate phthalate, hydroxypropylmethylcellulose phthalate, hydroxypropylmethyl acetate succinate (Shin-Etsu Chemicals), methacrylic copolymers (Rhon-Pharma, Eudragit® L-30D-55, L100-55, L100, 5100, etc.), etc. can be mentioned as examples of enteric coating agents. These materials are effective in terms of stability, even if they are directly used as enteric compositions.

The concentration or content of the therapeutic agent in the composition can be appropriately selected according to the physicochemical properties of the composition. When the composition is in a liquid form, the concentration can be about 0.0005 to about 30% (w/v) and preferably about 0.005 to about 25% (w/v). When the composition is a solid, the content can be about 0.01 to about 90% (w/w) and preferably about 0.1 to about 50% (w/w).

If necessary, additives such as a preservative (e.g. benzyl alcohol, ethyl alcohol, benzalkonium chloride, phenol, chlorobutanol, etc.), an antioxidant (e.g. butylhydroxyanisole, propyl gallate, ascorbyl palmitate, alpha- tocopherol, etc.), and a thickener (e.g. lecithin, hydroxypropylcellulose, aluminum stearate, etc.) can be used in the compositions and formulations as disclosed herein.

If necessary, one can use an emulsifier with the compositions and formulations as disclosed herein. This can be advantageous where, as, for example, with most vitamin A derivatives, the composition is fat soluble. Examples of emulsifiers that can be used include pharmaceutically acceptable phospholipids and nonionic surfactants. The emulsifiers can be used individually or in combinations of two or more. The phospholipid includes naturally occurring phospholipids, e.g. egg yolk lecithin, soya lecithin, and their hydrogenation products, and synthetic phospholipids, e.g. phosphatidylcholine, phosphatidylethanolamine, etc. Among them, egg yolk lecithin, soya lecithin, and phosphatidylcholine derived from egg yolk or soybean are preferred. The nonionic surfactant includes macro-molecular surfactants with molecular weights in the range of about 800 to about 20000, such as polyethylene-propylene copolymer, polyoxyethylene alkyl ethers, polyoxyethylene alkylarylethers, hydrogenated castor oil- polyoxyethylene derivatives, polyoxyethylene sorbitan derivatives, polyoxyethylene sorbitol derivatives, polyoxyethylene alkyl ether sulfate, and so on. When used, the proportion of the emulsifier is selected so that the concentration in a final administrable composition will be in the range of about 0.1 to about 10%, preferably about 0.5 to about 5%.

In addition to the above-mentioned components, a stabilizer for further improving the stability of the compositions and formulations as disclosed herein, such as an antioxidant or a chelating agent, an isotonizing agent for adjusting the osmolarity, an auxiliary emulsifier for improving the emulsifying power, and/or an emulsion stabilizer for improving the stability of the emulsifying agent can be incorporated. The isotonizing agent that can be used includes, for example, glycerin, sugar alcohols, monosaccharides, disaccharides, amino acids, dextran, albumin, etc. These isotonizing agents can be used individually or in combination, with two or more. An emulsion stabilizer that can be used, which includes cholesterol, cholesterol esters, tocopherol, albumin, fatty acid amide derivatives, polysaccharides, polysaccharide fatty acid ester derivatives, etc.

The compositions and formulations as disclosed herein can further comprise a viscogenic substance which can adhere to the digestive tract mucosa due to its viscosity expressed on exposure to water. Examples of such viscogenic substances include, but are not particularly limited as long as it is pharmaceutically acceptable, polymers (e.g. polymers or copolymers of acrylic acids and their salts) and natural-occurring viscogenic substances (e.g. mucins, agar, gelatin, pectin, carrageenin, sodium alginate, locust bean gum, xanthan gum, tragacanth gum, arabic gum, chitosan, pullulan, waxy starch, sucralfate, curdlan, cellulose, and their derivatives). Furthermore, for controling the release of the active drug or for formulation purposes, the additives conventionally used for preparing oral compositions can be added. Example of the additives include excipients (e.g. lactose, corn starch, talc, crystalline cellulose, sugar powder, magnesium stearate, mannitol, light anhydrous silicic acid, magnesium carbonate, calcium carbonate, L-cysteine, etc.), binders (e.g. starch, sucrose, gelatin, arabic gum powder, methylcellulose, carboxymethylcellulose, carboxymethylcellulose sodium, hydroxypropylcellulose, hydroxypropylmethylcellulose, polyvinylpyrrolidone, pullulan, dextrin, etc.), disintegrators (e.g. carboxymethylcellulose calcium, low-substituted hydroxypropylcellulose, croscarmellose sodium, etc.), anionic surfactants (e.g. sodium alkylsulfates etc.), nonionic surfactants (e.g. polyoxyethylene sorbitan fatty acid esters, polyoxyethylene fatty acid esters, polyoxyethylene-castor oil derivatives, etc.), antacids and mucous membrane protectants (e.g. magnesium hydroxide, magnesium oxide, aluminum hydroxide, aluminum sulfate, magnesium metasilicate aluminate, magnesium silicate aluminate, sucralfate, etc.), cyclodextrin and the corresponding carboxylic acid (e.g. maltosyl-beta-cyclodextrin, maltosyl-beta-cyclodextrin-carboxylic acid, etc.), colorants, corrigents, adsorbents, antiseptics, moistening agents, antistatic agents, disintegration retardants, and so on. The proportion of these additives can be appropriately selected from the range that can keep the stability and absorption of the basis.

The specific therapeutically effective dose level for any particular subject will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the compositions and formulations as disclosed herein which are employed; and like factors well known in the medical arts. For example, it is well within the skill of the art to either start doses of a compound at levels lower than required to achieve the desired therapeutic effect and to gradually increase the dosage until the desired effect is achieved, or start doses of a compound at high levels and to gradually decrease the dosage until the desired effect is achieved, as appropriate for the care of the individual patient.

The compositions as disclosed herein can also be administered in prophylactically or therapeutically effective amounts. A prophylactically or therapeutically effective amount means that amount necessary, at least partly, to attain the desired effect, or to delay the onset of, inhibit the progression of, or halt altogether, the onset or progression of the particular disease or disorder being treated. Such amounts will depend, of course, on the particular condition being treated, the severity of the condition and individual patient parameters including age, physical condition, size, weight and concurrent treatment. These factors are well known to those of ordinary skill in the art and can be addressed with no more than routine experimentation. It is preferred generally that a maximum dose be used, that is, the highest safe dose according to sound medical judgment. It will be understood by those of ordinary skill in the art, however, that a lower dose or tolerable dose can be administered for medical reasons, psychological reasons or for virtually any other reasons.

Efficacy of treatment can be monitored by the administering clinician. Where the disease or disorder is retinopathy of prematurity, the International Classification of Retinopathy or Prematurity (ICROP) can be applied. The ICROP uses a range of parameters to classify the disease. These parameters include location of the disease into zones (zones 1, 2 and 3), the circumferential extent of the disease based on clock hours 1-12, severity of the disease (stages 1-5), and the presence or absence of “Plus Disease.”

The zones are centered on the optic nerve. Zone 1 is the posterior zone of the retina, defined as the circle with a radius extending from the optic nerve to double the distance to the macula. Zone 2 is an annulus with the inner border defined by zone 1 and the outer border defined by the radius defined as the distance from the optic nerve to the nasal ora serrata. Zone 3 is the residual temporal crescent of the retina.

The circumferential extent of the disease is described in segments as if the top of the eye were 12 on the face of a clock. For example one might report that there is stage 1 disease for 3 clock hours from 4 to 7 o'clock.

The Stages describe the ophthalmoscopic findings at the junction between the vascularized and avascular retina.
Stage 1 is a faint demarcation line.
Stage 2 is an elevated ridge.
Stage 3 is extraretinal fibrovascular tissue.
Stage 4 is sub-total retinal detachment.
Stage 5 is total retinal detachment.

In addition, “Plus disease” may be present at any stage. “Plus disease” describes a significant level of vascular dilation and tortuosity observed at the posterior retinal vessels. This reflects the increase of blood flow through the retina (45).

Any improvement on the ICROP relative to pre-treatment classification is considered to be effective treatment. Similarly, where prevention of disease is the goal, treatment is considered effective if one or more signs or symptoms of ROP is less severe in a treated individual relative to the expected course of disease in a similar individual not receiving such treatment. The disease has been known and characterized to an extent that skilled clinicians can often predict the extent of disease that would occur in the absence of treatment, based, for example, on knowledge of earlier patients. The failure to develop or experience a worsening of one or more symptoms of ROP, or, for that matter any other retinal disease or disorder involving abnormal vascularization, can be considered effective prevention of disease in an individual otherwise expected to develop or experience worsening of such disease. Similarly, any improvement relative to expected disease state in the absence of treatment can be considered effective treatment.

As an alternative to the ICROP scale, other clinically accepted markers of retinal disease known to those of skill in the art can also be measured to monitor or determine the efficacy of treatment or prevention of retinal diseases or disorders as described herein. Generally a difference of at least 10% in a marker of retinal disease is considered significant.

It is understood that the foregoing detailed description and the following examples are illustrative only and are not to be taken as limitations upon the scope of the invention. Various changes and modifications to the disclosed embodiments, which will be apparent to those skilled in the art, may be made without departing from the spirit and scope of the present invention. Further, all patents, patent applications and publications cited herein are incorporated herein by reference.

The present invention may be as defined in any one of the following numbered paragraphs:

1. A method of improving rod-mediated retinal function, the method comprising administering to a subject with an immature retina, an agent that reduces rod energy demand, whereby rod-mediated retinal function is improved upon retinal maturity relative to a subject not treated with the agent.

2. The method of paragraph 1, wherein the subject is a premature infant.

3. The method of paragraph 1 or 2, wherein the subject is treated with supplemental oxygen.

4. A method of treating or preventing a retinal disease or disorder involving vascular abnormalities, the method comprising administering an agent that suppresses energy demand in rod photoreceptors of the eye.

5. The method of paragraph 4, wherein the treatment comprises administering N-retinylacetamide or a derivative thereof that suppresses energy demand in rod photoreceptors of the eye.

6. The method of paragraph 4, wherein the treatment is administered locally to the eye.

7. The method of paragraph 6, wherein the treatment is administered at a site distant from the eye.

8. The method of paragraph 4, wherein the retinal disease or disorder is retinopathy of prematurity.

9. The method of paragraph 4, wherein the retinal disease or disorder is selected from age-related macular degeneration and diabetic retinopathy.

10. A method for improving function and/or suppressing the visual cycle in a developing rod cell, the method comprising contacting the cell with an agent that suppresses energy demand in the rod cell.

11. The method of paragraph 10, wherein the treatment comprises contacting the rod cell with N-retinylacetamide or a derivative thereof.

12. Use of an agent that suppresses energy demand in rod photoreceptors of the eye for treatment of a developmental retinal disease or disorder in a subject.

13. The use of paragraph 12, wherein the subject is a premature infant.

14. The use of paragraph 12, wherein the subject is treated with supplemental oxygen.

15. The use of paragraph 12, wherein the agent is N-retinylacetamide.

16. The use of paragraph 12, wherein the agent is administered locally to the eye.

17. The use of paragraph 12, wherein the agent is administered at a site distant from the eye.

18. The use of paragraph 12, wherein the developmental retinal disease or disorder is selected from the group consisting of retinopathy of prematurity, age-related macular degeneration, and diabetic retinopathy.

19. Use of an agent that suppresses energy demand in rod photoreceptors of the eye for the preparation of a medicament for the treatment of a retinal disease or disorder in a subject.

20. The use of paragraph 19, wherein the subject is a premature infant.

21. The use of paragraph 19 wherein the subject is treated with supplemental oxygen.

22. The use of paragraph 19, wherein the agent is N-retinylacetamide.

23. The use of paragraph 19, wherein the agent is administered locally to the eye.

24. The use of paragraph 19, wherein the agent is administered at a site distant from the eye.

25. The use of paragraph 19, wherein the developmental retinal disease or disorder is selected from the group consisting of retinopathy of prematurity, age-related macular degeneration, and diabetic retinopathy.

### EXAMPLES

### Example 1.

### Reduction of Oxygen-induced Retinopathy Using N-Retinylacetamide.

### Purpose:

Rats with oxygen-induced retinopathy (OIR) are a common model of human retinopathy of prematurity (ROP). Both OIR and ROP are characterized by abnormal retinal vasculature and by lasting dysfunction of the neural retina. Recent findings in OIR rats imply a causal role for the rods in the ROP disease process. However, experimental manipulation of rod function is necessary to establish this role conclusively. The retinoid composition N-Retinylacetamide (all trans), of chemical composition C₂₂H₃₃NO “the drug” was administered in the rat OIR model of ROP.

### Methods:

OIR was induced in four litters of Sprague-Dawley pups (N=24) by exposure to alternating periods of 50% and 10% oxygen from the day of birth (P0) to P14. The light cycle was 12 hr light (10-30 lux) and 12 hr dark; the light-to-dark transition coincided with each oxygen alternation. For 15 days beginning P7, within one hour of this transition, the first and fourth litters were administered 6 mg/kg drug IP; the second and third litters received only vehicle. At P20-22, when marked retinal vascular abnormality is generally observed, electroretinograms were recorded and receptor and post-receptor function evaluated. Treatment effects were evaluated by ANOVA.

### Results:

Neither the maximal rod response nor the amplification constant of phototransduction were significantly changed by the drug treatment. However, the time-constant of deactivation of phototransduction, assessed by a double-flash protocol, was significantly shorter in drug-treated rats. Differences in post-receptoral responses were marked. While post-receptor sensitivity (log s) was unchanged, maximal scotopic b-wave amplitude was twice as large in drug-treated as vehicle-treated rats. The OFF response to a 400 ms stimulus presented in the presence of a background that suppressed the saturating a-wave by ˜90% was likewise 2×larger, as was the cone-driven response to 20 Hz flicker presented on the same background.

### Conclusions:

There was no evidence of serious, negative alteration of the photoreceptor response after treatment with N-Retinylacetamide. Importantly, the drug had a favorable effect on responses originating in the inner retina. The inner retina is supplied by the retinal vasculature; quantitative image analysis of fundus photographs can be used to determine the degree of vascular abnormality associated with OIR following such treatment. It is anticipated that the degree of vascular abnormality will be reduced in animals treated with the drug. Other drugs that target metabolic energy demand in the rod photoreceptor are anticipated to have similar effects.

### Example 2

### Down-regulation of the visual cycle favorably alters rod function in a rat model of ROP.

The effects of N-Retinylacetamide (Ret-NH2), a non-carotenoid vitamin-A derivative visual cycle modulator (VCM), were studied on rats with oxygen-induced retinopathy (OIR). Both OIR and ROP are characterized by lasting dysfunction of the neural retina and by abnormal retinal vasculature. Since the early status of the rods predicts blood vessel outcomes (J. D. Akula, et al., Invest Ophthalmol Vis Sci (2007) 48: 4351), it was suspected that the blood vessel abnormalities would also be altered.

Retinopathy was induced in Sprague-Dawley pups (N=46) by exposing them to alternating 24 hour periods of 50±1% and 10±1% oxygen from the day of birth to postnatal day (P) 14 as shown in FIG. 1. The light cycle was controlled at 12 hours 10-30 lux and 12 hours dark, except during test days when constant darkness was maintained. The light-to-dark transition was timed to coincide with each oxygen alternation. For two weeks, beginning on P7, during this transition, the first and fourth litters were injected intraperitoneally with 6 mg*kg-1 Ret-NH₂; the second and third litters were administered an equivalent volume of vehicle (20% dimethyl sulfoxide, DMSO) alone. The injection schedule was designed to continue over the age range that begins with the onset of rapid increase in the rhodopsin content of the retina and lasts until rhodopsin content exceeds 50% of its adult amount (A. B. Fulton, and B. N. Baker, Invest Ophthalmol Vis Sci (1984) 25:647). The dose was selected because studies in mouse suggested it would suppress the visual cycle by approximately 50%. The injection process resulted in the rats being held in room air (20.8% oxygen) for approximately 20 minutes between each oxygen alternation from P7-14. The rats were assessed following a longitudinal design with tests at P20-22, P30-32, and P60-62. These dates were selected because they capture the height of vascular abnormality, a period of marked recovery, and an adult age, respectively (J. D. Akula, et al., Invest Ophthalmol Vis Sci (2007) 48:4351). At each test age, we studied the function of the neural retina and the morphology of the retinal vasculature using noninvasive techniques.

Shortly (0-2 days) after the final dose, the effects of the drug were assessed on the neural retina by electroretinography (ERG). The timing and intensity of the stimuli, which was designed to assess rod photoreceptor and rod-mediated post-receptor neural function, were under computer control. Two sets of experiments were performed. In the first, rod and rod-mediated neural function in the dark-adapted retina were assessed. In the second, the recovery of the rod photoreceptor from a bright, rhodopsin-bleaching stimulus was assessed. Each set of experiments was performed on approximately half of the subjects from each litter.

Drawn from a rat in the first experiment (n=24), FIG. 2A shows a series of ERG a-waves elicited with flashes of doubling intensity, from one producing approximately 2,500 rhodopsin photoisomerizations per rod (R*) to one producing approximately 40,000 R*. Sample traces were recorded these from a P30-32 VCM-treated rat; the sample records in panels D, E, and G were recorded in the same session. Characteristics of the rod photo-response were estimated from the ERG by fitting the Hood and Birch (D. C. Hood, and D. G. Birch, Vis Neurosci (1992) 8:107) formulation of the Lamb and Pugh (T. D. Lamb, and E. N. Pugh, Jr., J Physiol (1992) 449:719; E. N. Pugh, Jr., and T. D. Lamb, Biochim Biophys Acta (1993) 1141:111) model of the biochemical processes involved in the activation of photo-transduction to the a-wave:

P₃(i,t)=Rmₚ₃·(1−exp(−½·i·S·(t−t_{d})²))

for td <t>20 ms. (1)

In this equation, i is the intensity of the flash (R*) and t is elapsed time (seconds). The free parameters in the model, Rmₚ₃, S, and t_{d}, were ensemble fitted using a square error minimization routine. Rmₚ₃ is the amplitude (μV) of the saturated rod response; it is proportional to the magnitude of the dark current and depends upon the number of channels available for closure by light in the ROS (T. D. Lamb, and E. N. Pugh, Jr., J Physiol (1992) 449:719; E. N. Pugh, Jr., and T. D. Lamb, Biochim Biophys Acta (1993) 1141:111). S is a sensitivity (R*-1*sec-2) parameter that is related to the amplification constant, A, which summarizes the kinetics of the series of processes initiated by the photoisomerization of rhodopsin and resulting in closure of the channels in the plasma membrane of the photoreceptor. td is a brief delay (sec). Fitting of the model was restricted to the leading edge of the a-wave. Analysis of variance (ANOVA) of the rod response sensitivity (S) data showed no statistically significant effect of VCM (FIG. 2B). On the other hand, the magnitude of the rod response (Rmₚ₃) was significantly increased by VCM (p<0.001), most markedly at the P30-32 test (FIG. 2C).

Following the study of the activation of phototransduction, deactivation of phototransduction was studied in the same rats using a double-flash paradigm. The time course of the rod response was derived to a green conditioning flash (CF) producing approximately 75 R*. This green flash, while producing an a-wave less than half of the saturated rod response, was nevertheless sufficient to fully suppress the dark current. First, the response to the CF alone was recorded. Then, the amplitude of the response to an intense, rod-saturating (approximately 2,000 R*) ‘white’ xenon-arc probe flash (PF, blue trace in FIG. 2A) was determined. The amplitude of the PF response, amax (i.iV), which was measured at 8 ms after presentation (just before the trough of the a-wave), was taken as proportional to the maximal rod dark current. Next, the CF and PF were presented together, separated by 10 predetermined inter-stimulus intervals (FIG. 2D). In double-flash conditions, the response to the CF recorded alone served as the baseline for measuring the amplitude of the response to the PF at each inter-stimulus time t, a_{sat,t}. The proportion of the dark current suppressed by the CF at elapsed time t, SFt, was, therefore, given by

$${SF}_{t} = 1 - \frac{{a}_{sat , t}}{{a}_{max}} . \qquad ( 2 )$$

To derive a value for the time-course of deactivation (FIG. 2E), the trough of the rod response was determined and fit a line through the recovery phase. The latency to 50% recovery, τ (ms) was noted. Recovery, as assessed by τ, was significantly (p<0.01) faster in VCM treated rats.

Rod-mediated post-receptor function was also assessed by evaluation of the ERG b-wave, using a series of ‘green’ flashes ranging from 0.07 to 300 R* to elicit 13 b-wave responses. FIG. 2G displays the first 8 responses, including to the first intensity at which the a-wave is clearly visible. The responses were fit to the Naka-Rushton function:

$$\frac{V ( i )}{Vm} = \frac{i}{i + σ} \qquad ( 3 )$$

as shown in FIG. 2H, only to those intensities before the appearance of a marked a-wave was noted at the higher end of the intensity range. In this equation, V(i) is the amplitude (μV) of the
response to a flash of i intensity (R*), Vm is the saturated amplitude (μV) of the b-wave, and σ the intensity (R*) that evokes a b-wave with amplitude of half Vm; again, if i is correctly specified, log σ is a measure of post-receptor sensitivity. As indicated by the filled circles in FIG. 2H, the function was fit only up to those intensities at which a-wave intrusion was first observed. Like S, log σ was not affected by VCM (FIG. 21), but Vm was significantly (p<0.01) increased in VCM rats (FIG. 2J).

In the second set of experiments, performed on cohorts (n=22), the recovery of the dark current was assessed from a 30 second light exposure estimated to bleach X% of rhodopsin. In this technique, the rod-saturating PF (2,000 R*) was presented to the dark-adapted eye, to determine the amplitude of the dark current. Next, a hemisected Ping-Pong ball was placed over the eye and the eye was stimulated with bright light to bleach the rhodopsin. Following the bleaching exposure, the response to the PF was assessed at two minute intervals for 40-50 minutes. At each time, the fraction of the dark current recovered (1-SFt) was calculated. The time to 50% recovery of the saturating rod photo-response, t₅₀, was assessed by fitting the Michaelis-Menten equation formulated as

$$RF ( t ) = 100 \% \cdot \frac{{t}^{n}}{{t}^{n} + {t}_{50}^{n}} \qquad ( 4 )$$

to the data, where RF(t) is the percentage of dark current recovered at time t after the bleach. Often, t₅₀ was longer than the recording session and was therefore extrapolated. t₅₀ was not significantly altered overall by VCM (FIG. 2L).

Ret-NH2 resulted in larger rod and rod-mediated response amplitudes (Rmp₃, Vm) and faster recovery from transduction (i), confirming that VCM significantly altered rod function. No unfavorable effects of rod function were found. Without wishing to be bound by theory, the larger rod response is best explained by lengthened ROS. If slowing the visual cycle resulted in less outer segment shedding, then the ROS would naturally elongate; this would have the effect of increasing the radial magnitude of the dark current. It would also serve to increase quantum efficiency of the rods, increasing the i term in eq. 1 and thus resulting in a proportionately lower calculated S, although not necessarily altering the underlying amplification constant, A; changes in rod sensitivity were not significant, but did trend lower at every age in the VCM rats. In addition, the ROS are disorganized in OIR (X. Reynaud, et al. (1995) Invest Ophthalmol Vis Sci 36, 2071).

Without wishing to be bound by theory, it may be that the slowed visual cycle merely allowed the rods to form better organized outer segments. Molecular motilities (especially of all-trans retinal trying the leave the ROS) would be faster in well formed outer segments. This would explain the reduction in τ in VCM rats. It might also account for the lack of a significant change in S: The greater photon capture in normally formed rods would be offset by an increase in A.

To summarize, these data are indicative of an advantageous, if transient, effect on rod function. Without wishing to be bound by theory, the transient enhancement of post-receptor function that also seems to have been affected by VCM administration may have resulted from preservation of synaptic connections in the outer plexiform layer which is attenuated in rat OIR models (A. Dorfman, et al., Invest Ophthalmol Vis Sci (2008) 49: 458).

To assess whether VCM treatment affected the retinal vasculature, wide-field images of the ocular fundus were obtained that show the major vessels of the retina following each ERG session. As shown in FIG. 3A, the images were composited to display a complete view of the posterior pole, the region within the circle bounded by the vortex veins and concentric to the optic nerve head, and the retinal region that in human patients is most important to the diagnosis of high-risk ROP. The arterioles were analyzed with RISA (R. Gelman, M. E. Invest Ophthalmol Vis Sci (2005) 46: 4734) custom image analysis software. In agreement with previous reports (J. D. Akula, Invest Ophthalmol Vis Sci (2007) 48:4351; K. Liu, et al., Invest Ophthalmol Vis Sci (2006) 47:2639), the venules were little affected by the induction of retinopathy. As shown in FIG. 3B, we analyzed all clear arterioles in each image. For each rat, the mean blood vessel integrated curvature across both eyes (median 10 arterioles) was determined to provide a measure of arteriolar tortuosity, TA (radians*pixel⁻¹), a measure which agrees well with subjective assessments in human subjects (R. Gelman, M. E. Invest Ophthalmol Vis Sci (2005) 46: 4734). As indicated in FIG. 3C, while there was no main effect of VCM on TA, a significant groupxage interaction (p=0.002) indicated that TA recovered more rapidly in VCM than vehicle treated rats. To test this, the value of TA at P20 was subtracted from the value of TA at P60 in each rat to determine the change in arteriolar tortuosity during the course of the experiment. An evaluation of these ΔP60-P20 data by t-test confirmed that VCM resulted in more than twice the normal amount of vascular recovery (p=0.001).

This is the first demonstration of a positive effect of systemic treatment with a VCM on pathology in an immature retina, in this case within the context of an animal model of ROP. Although our VCM regimen did not prevent the appearance of vascular abnormalities in the treated group, rod-mediated retinal function (both at the photoreceptor and in post-receptor neurons) was improved at the earliest test age (P20-22), and even more markedly improved at the second (P30-32), when drug would still have been affecting the system (M. Golczak, et al., Proc Natl Acad Sci USA (2005) 102: 8162). Thus, early neuro-protection led to greater vascular recovery. Future, adjusted VCM treatment regimens might improve this result. Nevertheless, these data demonstrate changes in rod function and retinal blood vessels in OIR. This supports a causal role for the rod photoreceptors in the developing retinal vasculature.

1. A method of improving rod-mediated retinal function, the method comprising administering to a subject with an immature retina, an agent that reduces rod energy demand, whereby rod-mediated retinal function is improved upon retinal maturity relative to a subject not treated with said agent.

2. The method of claim 1, wherein said subject is a premature infant.

3. The method of claim 1, wherein said subject is treated with supplemental oxygen.

4. A method of treating or preventing a retinal disease or disorder involving vascular abnormalities, the method comprising administering an agent that suppresses energy demand in rod photoreceptors of the eye.

5. The method of claim 4, wherein said treatment comprises administering N-retinylacetamide or a derivative thereof that suppresses energy demand in rod photoreceptors of the eye.

6. The method of claim 4, wherein said treatment is administered locally to the eye.

7. The method of claim 5, wherein said treatment is administered at a site distant from the eye.

8. The method of claim 4, wherein said retinal disease or disorder is retinopathy of prematurity.

9. The method of claim 4, wherein said retinal disease or disorder is selected from age-related macular degeneration and diabetic retinopathy.

10. A method for improving function and/or suppressing the visual cycle in a developing rod cell, the method comprising contacting said cell with an agent that suppresses energy demand in said rod cell.

11. The method of claim 10, wherein said treatment comprises contacting said rod cell with N-retinylacetamide or a derivative thereof.

12.-25. (canceled)

26. The method of claim 4, wherein the improved rod-mediated retinal function comprises a decrease in tortuosity compared to the tortuosity measured prior to treatment onset.

27. The method of claim 26, wherein tortuosity is determined using wide-field images of the ocular fundus.

28. The method of claim 4, wherein the rod-mediated retinal function is determined using electroretinography.

29. The method of claim 5, wherein the N-retinylacetamide is all trans N-retinylacetamide.

30. The method of claim 11, wherein the N-retinylacetamide is all trans N-retinylacetamide.
